(19) United States
(12) Patent Application Publication
(10) Pub. No.: US 2022/0208024 A1
(43) Pub. Date: Jun. 30, 2022

(54) DRIVE MECHANISM FOR A RESETTABLE MEDICAMENT DELIVERY TRAINING DEVICE

(71) Applicant: SHL MEDICAL AG, ZUG (CH)

(72) Inventor: Ling-Hsiang Chao, Taipei (TW)

(21) Appl. No.: 17/610,814

(22) PCT Filed: Jun. 8, 2020
(86) PCT No.: PCT/EP2020/065853

§ 371 Date: Nov. 12, 2021

(30) Foreign Application Priority Data
Jun. 26, 2019 (EP) 19182603.1

(51) Int. Cl. G09B 23/28

(52) U.S. Cl. CPC G09B 23/285

(57) ABSTRACT

A drive mechanism for a resettable medicament delivery training device is presented having a plunger rod having a proximal end and a distal end, a container having an inner chamber, an open proximal container end, and an open distal container end leading into the inner chamber, and a resetting structure, wherein in an initial state of the drive mechanism the plunger rod extends through the open distal container end into the inner chamber and is axially fixedly arranged in a first position in the container and biased towards the open proximal container end, and wherein the plunger rod is configured to be released from the first position, causing the plunger rod to move towards the open proximal container end to obtain a second position in the container, wherein the resetting structure is configured to be manually inserted into the container via the open proximal container end when the plunger rod is in the second position, the resetting structure being configured to be moved manually towards the distal open end of the container to reset the plunger rod in the first position.

### TECHNICAL FIELD

The present disclosure generally relates to medicament delivery devices. In particular, it relates to drive mechanism for a medicament delivery training device and to a medicament delivery training device comprising such a drive mechanism.

### BACKGROUND

Medicament delivery devices such as auto-injectors nowadays provide possibilities for the users themselves to handle medicament delivery in an easy, safe and reliable manner.

Before a user commences a drug administration programme by means of an auto-injector, it may be valuable for the user to undergo training to learn how to administer a drug properly by means of a specific auto-injector. A training device may be used for this purpose.

US2017/0069230 A1 discloses a resettable auto-injector training device. The resettable injection training device having an outer shell defining a chamber.

A plunger is slidable within the chamber. The device also includes a cap having an elongated rod that can be inserted into the device and slidably engageable relative to the device and can be used to reset the device after actuation, to prepare the device for a subsequent use. The cap has an elongated rod which is provided with a shoulder configured to interact with internal components of the device during resetting.

### SUMMARY

The resetting design disclosed in US2017/0069230 A1 is relatively complex. An object of the present disclosure is therefore to provide a drive mechanism for a medicament delivery training device which solves or at least mitigates problems of the prior art, easy to handle for a patient and none—expensive to manufacture.

There is hence according to a first aspect of the present disclosure provided a drive mechanism for a resettable medicament delivery training device, the drive mechanism comprising: a plunger rod having a proximal end and a distal end, a container having an inner chamber, an open proximal container end, and an open distal container end leading into the inner chamber, and a resetting structure, wherein in an initial state of the drive mechanism the plunger rod extends through the open distal container end into the inner chamber and is axially fixedly arranged in a first position in the container and biased towards the open proximal container end, and wherein the plunger rod is configured to be released from the first position, causing the plunger rod to move towards the open proximal container end to obtain a second position in the container, wherein the resetting structure is configured to be manually inserted into the container via the open proximal container end when the plunger rod is in the second position, the resetting structure being configured to be moved manually towards the distal open end of the container to reset the plunger rod in the first position.

A simple construction of the resettable drive mechanism may thereby be provided.

According to one embodiment the resetting structure is an elongated structure.

The resetting structure may have a constant cross-sectional area along a majority of an axial section of the resetting structure starting from its distal end tip to a midpoint of the resetting structure. The resetting structure may have a constant cross-sectional area along at least 70% or at least 80% or at least 90% of the length from the end tip to the midpoint.

According to one embodiment any cross-sectional dimension of that portion of the resetting structure which is arranged in the housing when the resetting structure is fully received by the housing may be smaller than a central opening of any internal component arranged inside the housing, including the housing. The portion of the resetting structure which is received by the housing may hence have no contact with any internal component other than the plunger rod inside the housing.

The resetting structure may be dimensioned such that it only interacts or cooperates with the plunger rod, in particular the stopper, when manually inserted into the housing.

When inserting the resetting structure into the housing, the resetting structure may first move through an open proximal housing end, and subsequently move only between radially inwards extending protrusions of the housing, which form a central proximal opening into the container, and an open proximal container end leading into the inner chamber.

One embodiment comprises a cap, wherein the resetting structure is fixedly arranged in the cap.

One embodiment comprises a holding structure provided with a radially flexible arm, wherein the plunger rod is provided with a radial recess configured to engage with the radially flexible arm to maintain the plunger rod axially fixed in the first position.

One embodiment comprises an actuator sleeve configured to receive the holding structure, the actuator sleeve being configured to move between a proximal position and a distal position, wherein in the proximal position the actuator sleeve is configured to urge the radially flexible arm radially inwards to engage with the radial recess.

According to one embodiment when the actuator sleeve is in the distal position the actuator sleeve is configured to enable the radially flexible arm to move radially outwards to disengage from the radial recess, thereby releasing the plunger rod from the first position.

According to one embodiment the actuator sleeve is biased towards the proximal position.

According to one embodiment the radially flexible arm is configured to engage with the radial recess when the plunger rod is moved from the second position towards the first position by the resetting structure, thereby axially locking the plunger rod in the first position.

One embodiment comprises a delivery member cover configured to move linearly relative to the container, between an initial position and an activating position, the delivery member cover being biased towards the initial position, wherein the delivery member cover is configured to move the actuator sleeve from the proximal position to the distal position when moved from the initial position to the activating position.

One embodiment comprises a housing configured to receive the container, wherein the housing has an open proximal housing end configured to receive the resetting structure.

According to one embodiment the housing has an inner stop structure configured to stop proximal movement of the plunger rod in the inner chamber, causing the plunger rod to obtain the second position.

According to one embodiment the inner stop structure comprises a plurality of radially inwards extending protrusions configured to enable the resetting structure to be received between them.

According to one embodiment the container is provided with a proximal engagement structure configured to engage with the housing.

According to one embodiment the radially inwards extending protrusions form a central proximal opening into the container, configured to receive the resetting structure.

According to one embodiment in an initial state of the delivery mechanism the stopper is arranged in the distal portion, the plunger rod being biased towards the proximal container end, and wherein the plunger rod is configured to be released from the first position, causing the plunger rod to move towards the proximal container end and the stopper to impact with a stopper contact surface, resulting in an initial signal, and to subsequently move into the proximal portion.

A medicament delivery training device, for which the drive mechanism is designed for, may thereby simulate medicament administration of a real medicament delivery device. In particular, a user is due to the initial signal able to learn and understand when the medicament administration process is commenced during medicament administration.

The initial signal may be an initial sound such as an initial click.

According to one embodiment the stopper has an outer stopper diameter, the distal portion has a distal portion inner diameter and the proximal portion has a proximal portion inner diameter which is smaller than the distal portion inner diameter and the outer stopper diameter, the distal portion transitioning to the proximal portion via the stopper contact surface. The stopper hence impacts with the stopper contact surface because the outer stopper diameter is larger than the proximal portion inner diameter.

One embodiment comprises a housing configured to receive the container, wherein the housing has an inner stop structure configured to stop proximal movement of the plunger rod in the inner chamber, causing the stopper to impact with the stop structure, resulting in a subsequent signal.

There is according to a second aspect of the present disclosure provided a resettable medicament delivery training device comprising a drive mechanism according to the first aspect.

Generally, all terms used in the claims are to be interpreted according to their ordinary meaning in the technical field, unless explicitly defined otherwise herein. All references to “a/an/the element, apparatus, component, means, etc. are to be interpreted openly as referring to at least one instance of the element, apparatus, component, means, etc.”, unless explicitly stated otherwise.

### BRIEF DESCRIPTION OF THE DRAWINGS

The specific embodiments of the inventive concept will now be described, by way of example, with reference to the accompanying drawings, in which:

FIG. 1 depicts a perspective view of an example of a medicament delivery training device;

FIG. 2 is an exploded view of the medicament delivery training device in FIG. 1;

FIG. 3 is a perspective view of a housing of the medicament delivery training device;

FIG. 4 is a perspective view of a housing of the medicament delivery training device when the container is fitted in the housing;

FIG. 5 is a longitudinal sectional view of a container of a medicament delivery training device;

FIG. 6 shows a perspective view of a plunger rod, an actuator sleeve and a holding structure;

FIG. 7 shows a perspective view of a cap with a resetting structure;

FIG. 8 is a longitudinal section of the medicament delivery training device in FIG. 1 in an initial state;

FIG. 9 is a longitudinal section of the medicament delivery training device with the cap removed;

FIG. 10 is a longitudinal section of the medicament delivery training device when the medicament delivery training device is being activated;

FIG. 11 is a longitudinal section of the medicament delivery training device when the plunger rod has been released;

FIG. 12 is a longitudinal section of the medicament delivery training device just before the plunger rod has moved maximally in the proximal direction;

FIG. 13 is a longitudinal section of the medicament delivery training device when the plunger rod has reached its most proximal position;

FIG. 14 is a longitudinal section of the medicament delivery training device when the delivery member cover has been released;

FIG. 15 is a longitudinal section of the medicament delivery training device when it is being reset; and

FIG. 16 is a longitudinal section of the medicament delivery training device when the medicament delivery training device has been reset.

### DETAILED DESCRIPTION

The inventive concept will now be described more fully hereinafter with reference to the accompanying drawings, in which exemplifying embodiments are shown. The inventive concept may, however, be embodied in many different forms and should not be construed as limited to the embodiments set forth herein; rather, these embodiments are provided by way of example so that this disclosure will be thorough and complete, and will fully convey the scope of the inventive concept to those skilled in the art. Like numbers refer to like elements throughout the description.

In the present disclosure, when the term “distal” is used, this refers to the direction pointing away from the dose delivery site. When the term “distal part/end” is used, this refers to the part/end of the medicament delivery training device, or the parts/ends of the members thereof, which under use of the medicament delivery training device is/are located furthest away from the dose delivery site. Correspondingly, when the term “proximal” is used, this refers to the direction pointing to the dose delivery site. When the term “proximal part/end” is used, this refers to the part/end of the medicament delivery training device, or the parts/ends of the members thereof, which under use of the medicament delivery training device is/are located closest to the dose delivery site.

Further, the term “longitudinal”, with or without “axis”, refers to a direction or an axis through the device or components thereof in the direction of the longest extension of the device and/or component.

Similarly, the terms “radial”, “transversal” or “orthogonal” refers to a direction generally perpendicular to the longitudinal direction which is the axis direction and e.g. “radially or orthogonally outward” will refer to a direction pointing away for the longitudinal axis.

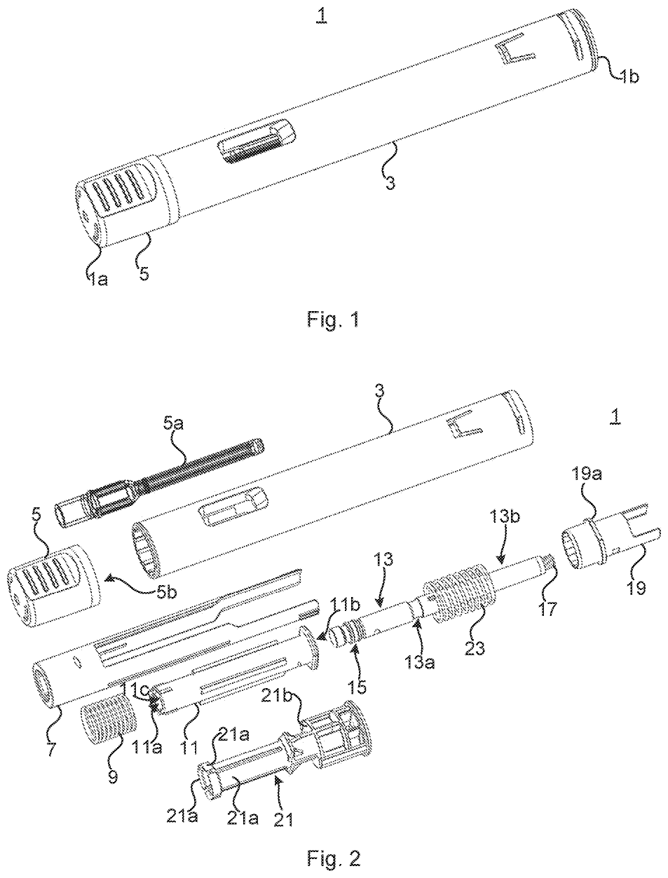

FIG. 1 depicts an example of a medicament delivery training device 1. The medicament delivery training device 1 is a dummy device to be used for learning how a corresponding real medicament delivery device is to be handled and operated. The medicament delivery training device 1 may be a medicament injection training device. The exemplified medicament delivery training device 1 is a resettable medicament delivery training device. The medicament delivery training device 1 is hence a multi-usage medicament delivery training device.

The medicament delivery training device 1 has a proximal end 1a and a distal end 1b. The medicament delivery training device 1 comprises a housing 3 and a cap 5. The cap 5 is configured to be removably attached to the housing 3. The cap 5 forms the proximal end of the medicament delivery training device 1 when attached to the housing 3.

FIG. 2 shows an exploded view of the medicament delivery training device 1. The exemplified medicament delivery training device 1 comprises a resetting structure 5a. The resetting structure 5a has an elongated shape. The resetting structure 5a may comprise a rod. The resetting structure 5a is configured to be attached to the cap 5. The cap 5 has a distal opening 5b configured to receive the resetting structure 5a. The resetting structure 5a extends in the distal direction from the cap 5 when fitted in the cap 5, along a central axis of the cap 5.

The medicament delivery training device 1 comprises a delivery member cover 7, and a first resilient member 9 configured to bias the delivery member cover 7 in the proximal direction. The exemplified first resilient member 9 may be a spring. The delivery member cover 7 is configured to be received by the housing 3. The delivery member cover 7 is configured to be moved linearly relative to the housing 3, between an initial position and an activation position. The delivery member cover 7 extends more in the proximal direction from the housing 3 in the initial position than in the activation position. Hereto, the initial position is an extended position and the activation position is a retracted position relative to the housing 3. The first resilient member 9 is configured to bias the delivery member cover 7 towards the initial position, i.e. the extended position.

The medicament delivery training device 1 comprises a container 11. The container 11 is a syringe dummy. Since it is a syringe dummy, the exemplified container 11 does not comprise any needle or cannula. The container 11 has an inner chamber 11a. The inner chamber 11a extends through the container 11. The container 11 has an open distal container end 11b. The container 11 has an open proximal container end 11c. The inner chamber 11a extends between the open distal container end 11b and the open proximal container end 11c.

The medicament delivery training device 1 comprises a plunger rod 13. The plunger rod 13 is provided with a stopper 15. The stopper 15 is provided on a proximal end of the plunger rod 13. The stopper 15 may be made of an elastic, resilient or flexible material.

The plunger rod 13 may have a proximal end face or cap/cover which may form part of the stopper 15 or which may extend in the proximal direction from the stopper 15. The proximal end face of the plunger rod 13 may be made of a rigid material such as a plastic material or a metal. In examples in which the plunger rod has a proximal end face, the proximal end face and the elastic stopper body together form the stopper as defined herein.

The plunger rod 13, the stopper 15 and the container 11 forms part of a drive mechanism of the medicament delivery training device 1.

The container 11 is configured to receive the plunger rod 13. The plunger rod 13 is configured to extend in the proximal direction into the inner chamber 11a through the open distal container end 11b.

The exemplified plunger rod 13 comprises a recess 13a. The recess 13a is a radial recess. In the example shown in FIG. 2, the recess 13a extends a full turn in the circumferential direction. The plunger rod 13 could alternatively comprise a plurality of physically separate recesses distributed in the circumferential direction. The recess 13a may have an inclined distal transition surface, relative to the central axis of the plunger rod 13, where the recess 13a transitions to the outer surface of the plunger rod 13.

The medicament delivery training device 1 comprises a second resilient member 17 configured to bias the plunger rod 13 in the proximal direction. The second resilient member 17 may be a spring. The second resilient member 17 is configured to be received by the plunger rod 13. The plunger rod 13 may hence be hollow.

The medicament delivery training device 1 comprises a holding structure 21. The holding structure 21 is configured to be received by the housing 3. The holding structure 21 is configured to be fixedly arranged in the housing 3. The holding structure 21 is axially fixed relative to the housing 3. The holding structure 21 is arranged in a distal portion of the housing 3. The holding structure 21 is an elongated structure. The holding structure 21 is hollow. The holding structure 21 is configured to receive a distal end portion 13b of the plunger rod 13.

The holding structure 21 comprises a plurality of radially flexible arms 21a. The radially flexible arms 21a extend in the proximal direction. The radially flexible arms 21a form a proximal end of the holding structure 21. Each radially flexible arm 21a is configured to engage with the recess 13a of the plunger rod 13. Each radially flexible arm 21a comprises a radially inwards extending protrusion configured to engage with the recess 13a.

The medicament delivery training device 1 comprises an actuator sleeve 19. The actuator sleeve 19 is configured to be arranged around a proximal portion of the holding structure 21. The actuator sleeve 19 is configured to be moved linearly relative to the holding structure 21 between a proximal position and a distal position. The delivery member cover 7 is configured to move the actuator sleeve 19 from the proximal position to the distal position. When the delivery member cover 7 is in the initial position, the actuator sleeve 19 is arranged in the proximal position. When the delivery member cover 7 is moved in the distal direction towards the activation position, the actuation sleeve 19 is moved towards the distal position. The actuator sleeve 19 has a proximal flange 19a. The delivery member cover 7 has distally extending legs configured to bear against/engage with the proximal flange 19a when the delivery member cover 7 is moved towards the activation position. The actuation sleeve 19 is thereby moved towards the distal position as the delivery member cover 7 is moved in the distal direction.

The actuator sleeve 19 is configured to be arranged around the radially flexible arms 21a and urge the radially flexible arms 21a to engage with the recess 13a of the plunger rod 13 when the actuator sleeve 19 is in the proximal position.

The medicament delivery training device 1 comprises a third resilient member 23. The third resilient member 23 may be a spring. The third resilient member 23 is configured to bias the actuator sleeve 19 in the proximal direction towards the proximal position. The holding member 21 has a distal flange surface 21b. The third resilient member 23 is configured to be arranged between the distal end of the proximal flange 19a of the actuator sleeve 19 and the distal flange surface 21b. The third resilient member 23 thereby biases the actuator sleeve 19 in the proximal direction towards the proximal position.

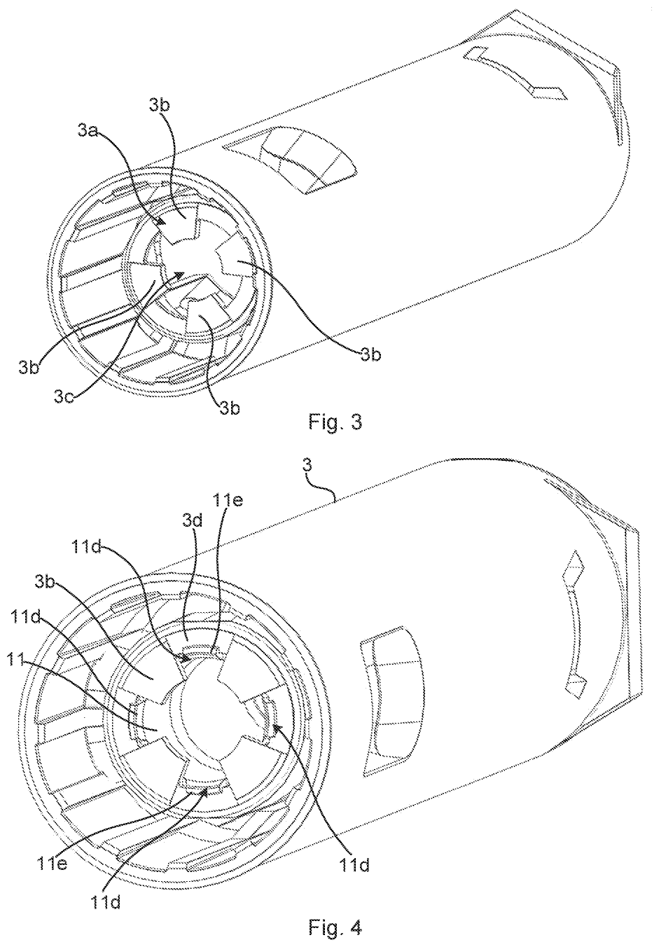

FIG. 3 shows a perspective view of the housing 3. The housing 3 has an inner stop structure 3a. The stop structure 3a comprises a plurality of radially inwards extending protrusions 3b. The radially inwards extending protrusions 3b form a central proximal opening 3c between them. The radially inwards extending protrusions 3b do hence not meet as they extend towards the central axis of the housing 3. The central proximal opening 3c is dimensioned such that the resetting structure 5a may be received through the central proximal opening 3c when the cap 5 with the resetting structure 5a is attached to the housing 3 and hence the resetting structure 5a is inserted into the housing 3. The central proximal opening 3c is dimensioned such that the stopper 15 is not able to move through the central proximal opening 3c when the plunger rod 13 is moved in the proximal direction, as will be explained in what follows. Hereto, the stopper has an outer stopper diameter which is larger than the central proximal opening 3c.

FIG. 4 shows a perspective view of the housing 3 when the container 11 is arranged in the housing 3. The container 11 is provided with a proximal engagement structure 11d configured to engage with the housing 3. The proximal engagement structure 11d comprises a plurality of radially outwards extending flexible snap structures 11e. According to the present example, the housing 3 has a radially inwards extending structure 3d between each adjacent pair of radially inwards extending protrusion 3b. The radially inwards extending structures 3d have a shorter radially inwards extension than the radially inwards extending protrusions 3b. Each of the plurality of radially outwards extending flexible snap structures 11e is configured to engage with a respective radially inwards extending structure 3d. The container 11 is thereby attached to the housing 3.

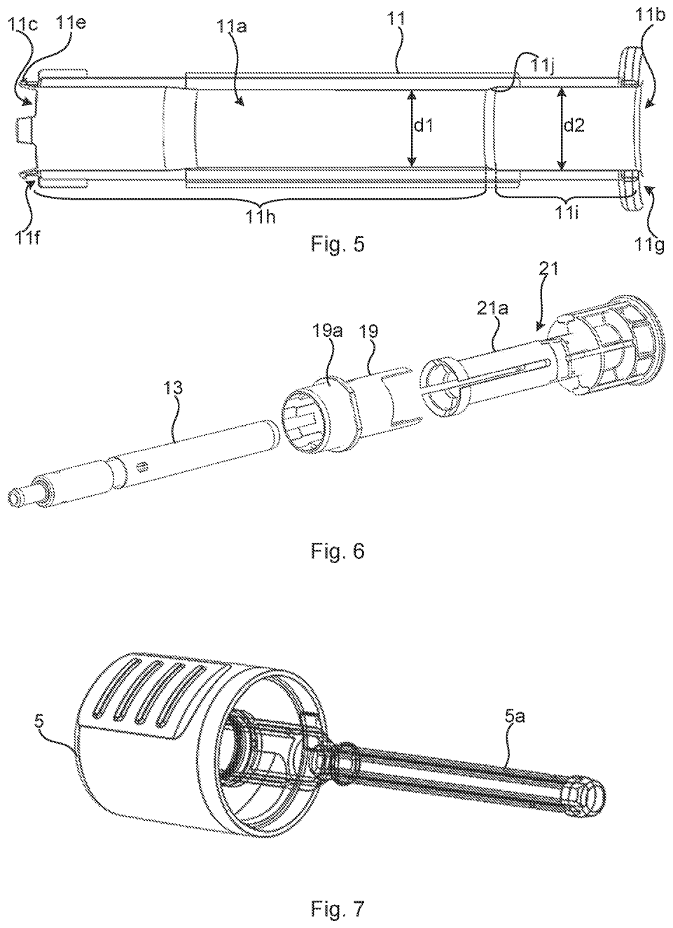

FIG. 5 shows a longitudinal sectional view of the container 11. The container 11 has a proximal container end 11f and a distal container end 11g. The inner chamber or channel 11a which extends between the proximal container end 11f and the distal container end 11g has a proximal portion 11h and a distal portion 11i. The proximal portion has a proximal portion inner diameter d1 and the distal portion 11i has a distal portion inner diameter d2. The proximal portion inner diameter d1 is smaller than the distal portion inner diameter d2.

The open distal container end 11b opens into the distal portion 11i. The distal portion 11i transitions to the proximal portion 11h via a stopper contact surface 11j. The stopper contact surface 11j may for example be inclined relative to the central longitudinal axis of the container 11, as shown in FIG. 5, or it may be orthogonal to the central longitudinal axis. In the former case, the stopper contact surface 11j provides a gradual decrease of the inner diameter of the container 11 in the proximal direction. In the latter case, the stopper contact surface forms a step between the distal portion 11i and the proximal portion 11h.

The outer stopper diameter may for example be defined by the largest outer dimension or diameter of the stopper 15. The proximal portion inner diameter d1 is smaller than the outer stopper diameter. The proximal portion inner diameter d1 is dimensioned to provide friction onto the stopper 15 to control the driving speed of the plunger rod 13. The driving speed should preferably simulate the driving speed of the plunger rod of the corresponding real medicament delivery device which the medicament delivery training device 1 is designed to simulate.

FIG. 6 shows a perspective view of the plunger rod 13, the actuator sleeve 19 and the holding structure 21.

FIG. 7 is a perspective view of the cap 5 with the resetting structure 5a attached to the cap 5. In a typical example, the resetting structure 5a is fixedly attached to the cap 5 such that when the cap 5 is removed from the housing 3, the resetting structure 5a is also removed from the housing 3 together with the cap 5.

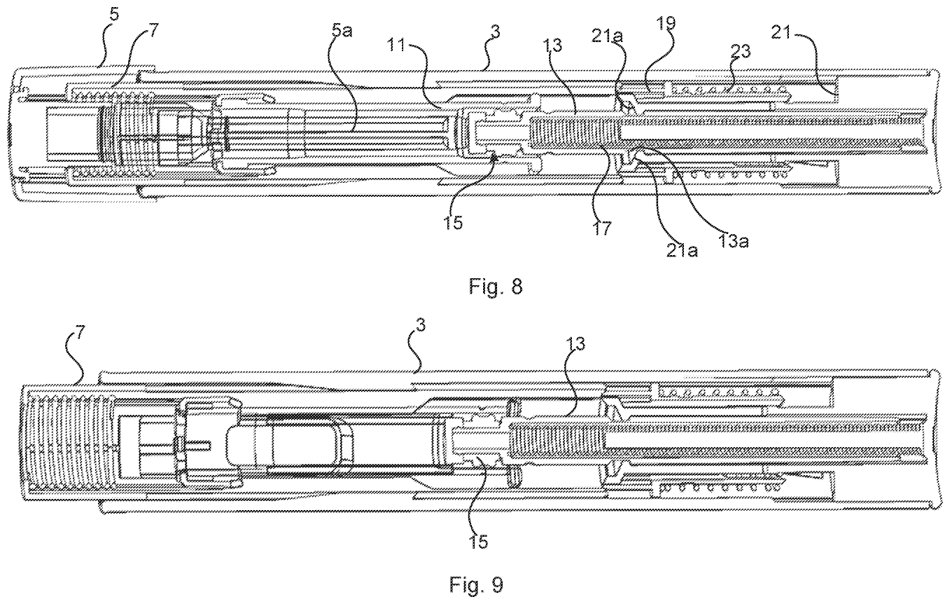

FIG. 8 shows a longitudinal section of the medicament delivery training device 1 in an initial state thereof. The initial state is the state of the medicament delivery training device 1 before it has been activated. In the initial state, the cap 5 is attached to the housing 3. The resetting structure 5a extends into the container 11 through the central proximal opening 3c of the housing 3. The drive mechanism is also in an initial state when the medicament delivery training device 1 is in the initial state.

The actuator sleeve 19 is arranged in the proximal position as it is biased by the third resilient member 23 in the proximal direction and the delivery member cover 7 is in the initial position in which it does not apply any, or at least negligible force in the distal direction onto the actuator sleeve 19. The radially flexible arms 21a are hence urged radially inwards by the actuator sleeve 19 which is arranged around the radially flexible arms 21a. The radially flexible arms 21a thereby engage with the recess 13a. The plunger rod 13 is therefore maintained in a first position in which it extends through the open distal container end 11b and the stopper 15 is arranged fixed in the distal portion 11i of the container 11. The proximal end of the stopper 15 is typically arranged at a distance from the stopper contact surface 11j.

When a user is to perform training with the medicament delivery training device 1, the cap 5 is first removed from the housing 3. This is shown in FIG. 9. As the cap 5 is removed, so is the resetting structure 5a. The delivery member cover 7 is in the initial position, i.e. in the extended position relative to the housing 3. The plunger rod 13 is still fixed in the first position as in the initial state depicted in FIG. 8.

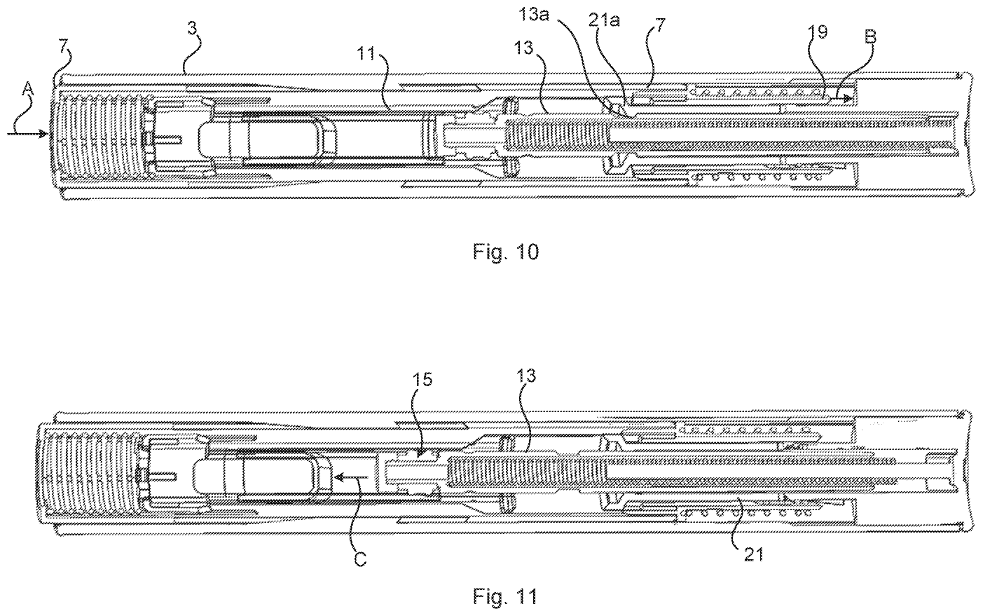

In FIG. 10, the delivery member cover 7 has been moved into the housing 3 to the activation position as shown by arrow A. This may for example be achieved by a user pressing the medicament delivery training device 1 towards an intended site of injection. The delivery member cover 7 has pushed the actuation sleeve 19 in the distal direction towards the distal position, as shown by arrow B. The actuation sleeve 19 is hence moved distally from the radially flexible arms 21a. Since the plunger rod 13 is biased in the proximal direction and urged towards the proximal container end 11c, the radially flexible arms 21a are now moved out from the recess 13a. The plunger rod 13 is hence released and moved in the proximal direction further into the container 11.

FIG. 11 shows the medicament delivery training device 1 when the plunger rod 13 has been released from its engagement with the holding structure 21. The stopper 15 has moved from the distal portion 11i into the proximal portion 11h of the inner chamber 11a via the stopper contact surface 11j, as shown by arrow C. As the stopper 15 is moved from the distal portion 11i into the proximal portion 11h, the stopper impacts with the stopper contact surface 11j, since the outer stopper diameter is larger than the proximal portion inner diameter d1. This impact results in an initial signal. The initial signal may be an audible sound such as a click. A user will thereby become aware that simulated medicament administration has commenced.

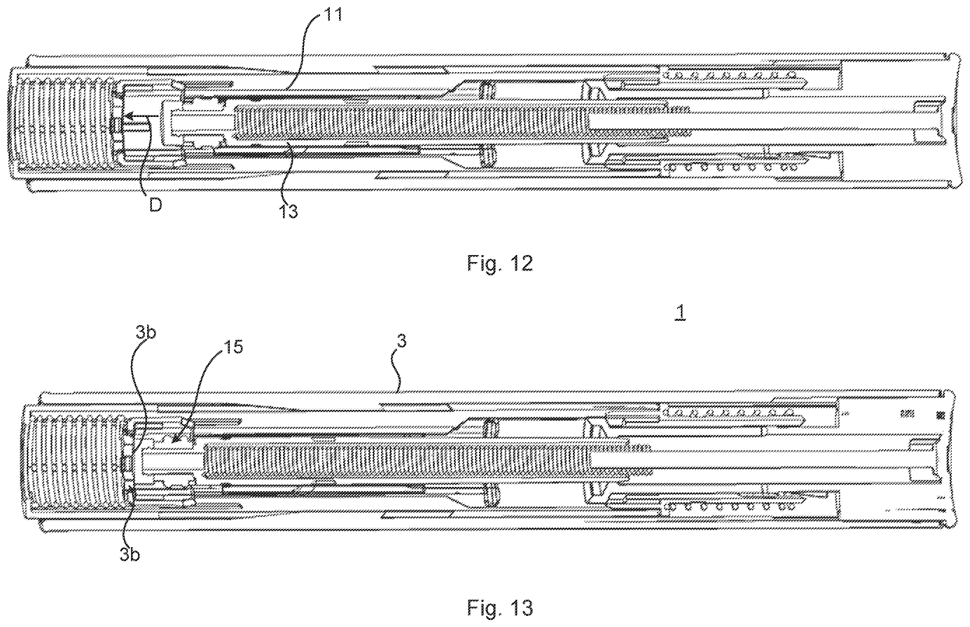

In FIG. 12 the plunger rod 13 has been further moved in the proximal direction inside the container 11 as shown by arrow D.

In FIG. 13, the plunger rod 13 is in a second position and the stopper 15 has impacted with the housing 3, in particular with the stop structure 3a and the plurality of radially inwards extending protrusions 3b. A subsequent signal is generated by this impact. The subsequent signal may for example be an audible sound such as a click. A user of the medicament delivery training device 1 will thereby understand that the simulated medicament injection has been finalised.

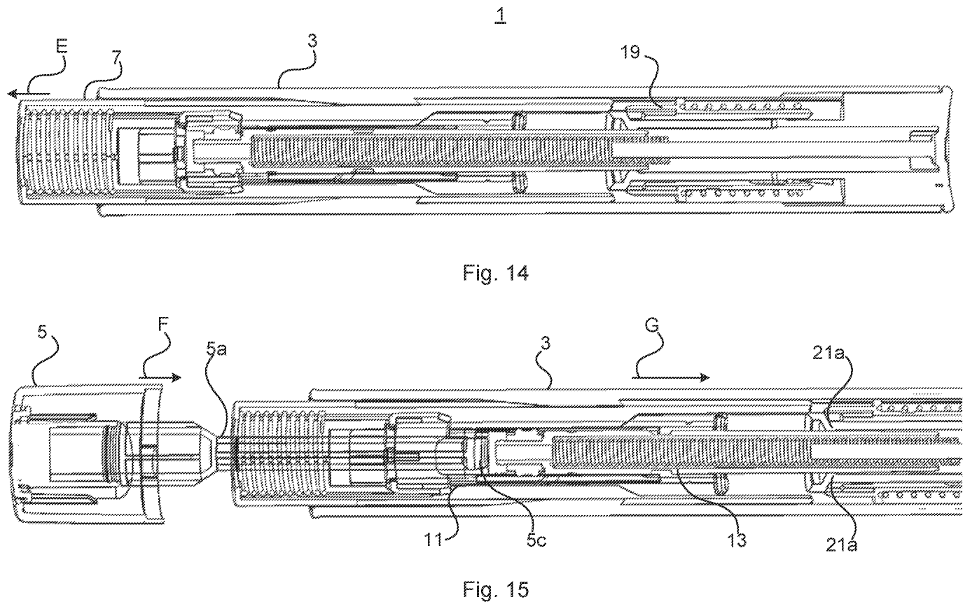

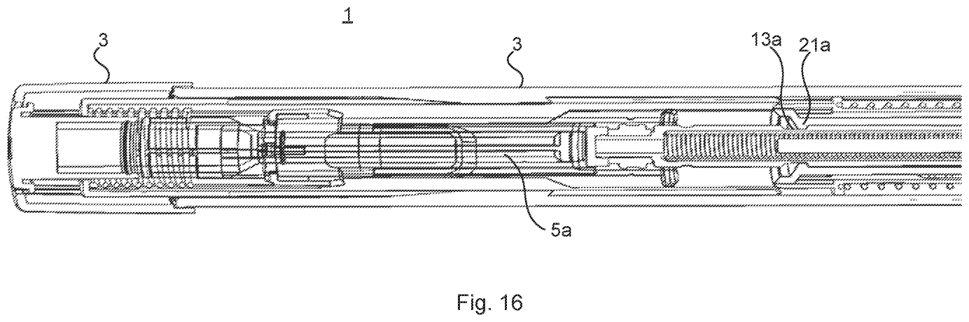

In FIG. 14 the medicament delivery training device 1 has been removed from the injection site by the user. The delivery member cover 7 is hence moved back to the initial position due to its proximal biasing, as shown by arrow E. As a result, the actuation sleeve 19 is moved back to the proximal position from the distal position.

In this used state of the medicament delivery training device 1, the medicament delivery training device 1 may be reset manually for further training. The plunger rod 13 may in particular be reset to the first position. This resetting may be performed by manually inserting the resetting structure 5a. In particular, the resetting may be performed manually by a user placing the cap 5 back onto the housing 3.

FIG. 15 shows a resetting operation of the medicament delivery training device 1. The cap 5 is being placed back onto the housing 5 as shown by arrow F. The resetting structure 5a thereby first penetrates the central proximal opening 3c formed between the radially inwards extending protrusions 3b and subsequently the open proximal container end 11c of the container 11. The resetting structure 5a has a distal end tip Sc which pushes the plunger rod 13 in the distal direction towards the first position, as shown by arrow G, when the resetting structure 5a is moved in the distal direction inside the container 11. The second resilient member 17 is thereby compressed. The resetting structure 5a has a length which enables it to reset the plunger rod 13 in the first position when the cap 5 is attached to the housing 3. Hence, as the resetting structure 5a is moved further into the container 11 the recess 13a of the plunger rod 13 will become aligned with the radially flexible arms 21a, which at this time are urged radially inwards by the actuator sleeve 19 arranged in the proximal position and hence around the radially flexible arms 21a. The radially flexible arms 21a will therefore engage with the recess 13a and the plunger rod 13 once again becomes fixedly arranged in the first position with the stopper 15 arranged in the distal portion 11i of the container 11. This is shown in FIG. 15, where the medicament delivery training device 1 has once again obtained its initial state in which it is ready to be used for further training.

The inventive concept has mainly been described above with reference to a few examples. However, as is readily appreciated by a person skilled in the art, other embodiments than the ones disclosed above are equally possible within the scope of the inventive concept, as defined by the appended claims.

1-15. (canceled)

16: A drive mechanism for a resettable medicament delivery training device, the drive mechanism comprising:
a plunger rod having a proximal end and a distal end,
a container having an inner chamber, an open proximal container end, and an open distal container end leading into the inner chamber,
and a resetting structure,
wherein in an initial state of the drive mechanism the plunger rod extends through the open distal container end into the inner chamber and is axially fixedly arranged in a first position in the container and biased towards the open proximal container end, and wherein the plunger rod is configured to be released from the first position, causing the plunger rod to move towards the open proximal container end to obtain a second position in the container,
wherein the resetting structure is configured to be manually inserted into the container via the open proximal container end when the plunger rod is in the second position, the resetting structure being configured to be moved manually towards the distal open end of the container to reset the plunger rod in the first position.

17: The drive mechanism as claimed in claim 16, wherein the resetting structure is an elongated structure.

18: The drive mechanism as claimed in claim 16, comprising a cap, wherein the resetting structure is fixedly arranged in the cap.

19: The drive mechanism as claimed in claim 16, comprising a holding structure provided with a radially flexible arm, wherein the plunger rod is provided with a radial recess configured to engage with the radially flexible arm to maintain the plunger rod axially fixed in the first position.

20: The drive mechanism as claimed in claim 19, comprising an actuator sleeve configured to receive the holding structure, the actuator sleeve being configured to move between a proximal position and a distal position, wherein in the proximal position the actuator sleeve is configured to urge the radially flexible arm radially inwards to engage with the radial recess.

21: The drive mechanism as claimed in claim 20, wherein when the actuator sleeve is in the distal position the actuator sleeve is configured to enable the radially flexible arm to move radially outwards to disengage from the radial recess, thereby releasing the plunger rod from the first position.

22: The drive mechanism as claimed in claim 20, wherein the actuator sleeve is biased towards the proximal position.

23: The drive mechanism as claimed in claim 20, wherein the radially flexible arm is configured to engage with the radial recess when the plunger rod is moved from the second position towards the first position by the resetting structure, thereby axially locking the plunger rod in the first position.

24: The drive mechanism as claimed in claim 20, comprising a delivery member cover configured to move linearly relative to the container, between an initial position and an activating position, the delivery member cover being biased towards the initial position, wherein the delivery member cover is configured to move the actuator sleeve from the proximal position to the distal position when moved from the initial position to the activating position.

25: The drive mechanism as claimed in claim 16, comprising a housing configured to receive the container, wherein the housing has an open proximal housing end configured to receive the resetting structure.

26: The drive mechanism as claimed in claim 25, wherein the housing has an inner stop structure configured to stop proximal movement of the plunger rod in the inner chamber, causing the plunger rod to obtain the second position.

27: The drive mechanism as claimed in claim 26, wherein the inner stop structure comprises a plurality of radially inwards extending protrusions configured to enable the resetting structure to be received between them.

28: The drive mechanism as claimed in claim 25, wherein the container is provided with a proximal engagement structure configured to engage with the housing.

29: The drive mechanism as claimed in claim 27, wherein the radially inwards extending protrusions form a central proximal opening into the container, configured to receive the resetting structure.

30: A resettable medicament delivery training device comprising a drive mechanism as claimed in claim 16.

31: An assembly for a resettable medicament delivery training device, the assembly comprising:
a drive mechanism comprising;
a hollow plunger rod having a proximal end and a distal end;
a spring partially positioned within the plunger rod; and
a holding structure releasably engaged with an outside surface of the plunger rod,
a container having an inner chamber, an open proximal end, and an open distal end; and
a resetting structure,
wherein in an initial state of the holding structure the plunger rod extends through the open distal end and is axially fixedly arranged in a first position relative to the container such that the spring biases the plunger rod towards the open proximal,
wherein movement of the holding structure from the initial state disengages the plunger rod from the first position such that the plunger rod moves axially towards the open proximal end to reach a second position relative to the container, and
wherein the resetting structure is manually inserted into the open proximal end when the plunger rod is in the second position such that the resetting structure is moved manually towards the distal open end so as to push and reset the plunger rod back to the first position.

32: The assembly of claim 31, wherein the resetting structure is fixedly arranged in a cap releasably connectable to a housing that holds the drive mechanism.

33: The assembly of claim 31, wherein the holding structure comprises a radially flexible arm releasably engaged with a radial recess on the plunger rod to maintain the plunger rod axially fixed in the first position.

34: The assembly of claim 33, further comprising an actuator sleeve operationally engaged with the holding structure having a radially flexible arm, where the actuator sleeve moves between a proximal position and a distal position, wherein when in the proximal position the actuator sleeve prevents the radially flexible arm from flexing radially outward and disengaging from the radial recess.

35: The assembly of claim 33, further, wherein the housing has an inner stop structure with a through hole and configured to stop proximal movement of the plunger rod relative to the container as the plunger rod moves from the first position to the second position.
